(19) United States
(12) Patent Application Publication
(10) Pub. No.: US 2008/0183218 A1
(43) Pub. Date: Jul. 31, 2008

(54) System and Methods for Spinous Process Fusion

(75) Inventors: Rich Mueller, Carlsbad, CA (US); Benjamin Arnold, La Jolla, CA (US); Eric Dasso, San Diego, CA (US)

Correspondence Address: JONATHAN SPANGLER;NU VASIVE, INC., 4545 TOWNE CENTRE COURT, SAN DIEGO, CA, 92121, US

(73) Assignee: NuVasive, Inc., San Diego, CA (US)

(21) Appl. No.: 12/024,025

(22) Filed: Jan. 31, 2008

Related U.S. Application Data
(60) Provisional application No. 60/898,818, filed on Jan. 31, 2007.

(51) Int. Cl. A61B 17/58; A61B 17/56

(52) U.S. Cl. 606/280; 606/300

(57) ABSTRACT

A spinal fixation device including two plates and a coupling element for coupling the plates in a fixed manner about adjacent spinous processes of the spine. Each plate is preferably equipped with integral spikes on the inwardly facing surfaces for pressing into the spinal processes and thereby augmenting the purchase between the plates and the spinous processes. Each plate contains a central aperture through which the coupling element passes in order to couple the plates together.

### CROSS REFERENCES TO RELATED APPLICATIONS

The present application is a nonprovisional patent application claiming benefit under 35 U.S.C. § 119(e) from U.S. Provisional Application Ser. No. 60/898,818, filed on Jan. 31, 2007, the entire contents of which are hereby expressly incorporated by reference into this disclosure as if set forth fully herein.

### BACKGROUND OF THE INVENTION

I. Field of the Invention

The present invention relates generally to spinal surgery, and more particularly to devices for fusing adjacent spinous processes to stabilize the vertebral segment associated with the particular spinous processes.

II. Discussion of the Prior Art

The human spinal column is made up of two basic components, vertebrae (bone) and intervertebral discs (gel-like cushions that absorb pressure and prevent vertebrae from rubbing together). A number of vertebrae and intervertebral discs stack together to form a column that provides support and structure for the body while still allowing a large degree of motion and flexibility. The spinal column also serves to protect the spinal cord (a bundle of nerves linking the brain to the rest of the body) that runs through an opening formed in the center of the column. A pair of nerve roots exit the spinal column at each level through spaces formed between the vertebrae. Various traumatic events and degenerative conditions may result in undesirable motion or changes in disc height, both of which may cause chronic pain for the affected individual. The pain is generally caused when changes in disc height and improper motion allow adjacent vertebrae to impinge upon exiting nerve roots. The degree and treatment of pain varies by individual but in many instances the pain can be disabling and uncontrollable by non-invasive means, leaving surgery as the only viable option. Generally in such a case, two or more vertebrae are fused together, employing various instrumentation and methods to correct disc height and prevent improper movement of the vertebrae while fusion occurs, thereby eliminating or at least reducing the pain of the affected individual.

While there are a variety of systems and methods for effecting spinal fixation while fusion occurs, one of the more common methods involves securing pedicle screws into the pedicles of the two or more adjacent vertebrae to be fixed. The challenge in this method is securing the pedicle screws without breaching, cracking, or otherwise compromising the pedicle wall, which may occur if the screw is not properly aligned with the pedicle axis. If the pedicle (or more specifically, the cortex of the medial wall, lateral wall, superior wall and/or inferior wall) is breached, cracked, or otherwise compromised, the patient may experience pain or neurological deficit due to unwanted contact between the pedicle screw and delicate neural structures, such as the spinal cord or exiting nerve roots. This may necessitate revision surgery, which is disadvantageously painful for the patient and costly, both in terms of recovery time and hospitalization.

The present invention is directed to overcome one or more shortcomings encountered with current fixation devices and systems.

### SUMMARY OF THE INVENTION

The present invention relates to a spinal fixation device designed to be attached to adjacent spinous processes of the spine for immobilizing the adjacent spinous processes to promote fusion therebetween. The spinal fixation device may be used alone (that is, without any supplemental fusion devices, such as interbody fusion implants) or with supplemental fixation devices. In either event, the spinal fixation device allows fusion to occur between the adjacent spinous processes by maintaining them in an immobilized, locked relationship such that a boney bridge can form therebetween. The formation of the fusion bridge between the adjacent spinous processes may be augmented or facilitated by placing fusion-enhancing compounds between the spinous processes, including but not limited to autologous bone, allograft bone, bone morphogenic protein (BMP), and/or any number of suitable biomaterials.

According to one embodiment of the present invention, the spinal fixation device includes two plates and a coupling element for coupling the plates in a fixed manner about adjacent spinous processes of the spine. Each plate is preferably equipped with integral spikes on the inwardly facing surfaces for pressing into the spinal processes and thereby augmenting the purchase between the spinous processes and the plates. Each plate contains a central aperture through which the coupling element passes in order to couple the plates together.

The coupling element may be any number of devices capable of coupling the first plate to the second plate. In one exemplary embodiment, the coupling element may be a screw or bolt with one end threaded to engage a threaded aperture in one plate and the other end with a head dimensioned to engage with a respective region on the other plate and a driving tool. In another embodiment, the threaded end of the coupling element may be replaced with one having external ridges (as opposed to threads) to engage corresponding features in the aperture of one plate to prevent any backward motion once received through the aperture. This embodiment is advantageous in that the plates can be easily locked together and tightened by simply pushing the coupling element through one plate (with the head received within a corresponding region or recess of the first plate) and into the next (with the ridges locking at each point as the ridged section is advanced through the aperture of the second plate, the head may or may not be fully contained within the first plate). In either embodiment, the head may be constructed like a screw head with an internally disposed recess for receiving a driving element (e.g. hexalobe drive, Phillips screw driver, hex driver, etc. . . . ) or may be constructed without such an internally disposed recess and may instead be driven by an exteriorly placed driving element (e.g. wrench).

The apertures may be provided in any number of different manners to help facilitate coupling the fixation element to the plates. For example, with the first embodiment of the coupling element (threaded screw or bolt), the aperture of one plate may be tapped with internal threads to engage external threads of the threaded section of the coupling element. With the second embodiment of the coupling element (ridged bolt), the aperture of one plate may be equipped with any number of suitable features, such as inwardly facing teeth or ridges, that engage with the ridges of the coupling element.

Any number of suitable instruments may be provided to help facilitate the surgery, including but not limited to instruments for compressing and/or distracting the adjacent spinous processes prior to securing the plates (and thus immobilizing the spinous processes), as well as instruments to facilitate coupling the plates together such as drivers for tightening the coupling element to the plates or instruments for compressing the plates together. In one embodiment, the driving or compressing instrument may be equipped with a torque limiting mechanism that produces an audible (e.g. “click”) and/or and a tactile alert that lets the surgeon know he or she has applied optimal torque to the fixation element to fix the plates together.

### BRIEF DESCRIPTION OF THE DRAWINGS

FIG. 1 is a postero-lateral view of a portion of the spine with one example of a spinous process fixation system according to a first embodiment of the present invention implanted on adjacent spinous processes of a spine;

FIG. 2 is a posterior view of the spinous process fixation system implanted on adjacent spinous processes as shown in FIG. 1;

FIG. 3 is a perspective view of the spinous process fixation system of FIG. 1;

FIG. 4A is a plan view of the lateral surface of a first plate forming part of the spinous process fixation system of FIG. 3;

FIG. 4B is a plan view of the medial surface of the first plate of FIG. 4A;

FIG. 5A is a plan view of the lateral surface of a second plate forming part of the spinous process fixation system of FIG. 3;

FIG. 5B is a plan view of the medial surface of the second plate of FIG. 5A;

FIG. 6 is a perspective view of one example of a coupling element forming part of the spinous process fixation system of FIG. 3;

FIG. 7 is a postero-lateral view of a portion of the spine with one example of a spinous process fixation system according to a second embodiment of the present invention implanted on adjacent spinous processes of a spine;

FIG. 8 is a posterior view of the spinous process fixation system implanted on adjacent spinous processes as shown in FIG. 7;

FIG. 9 is a perspective view of the spinous process fixation system of FIG. 7;

FIG. 10A is a plan view of the lateral surface of a first plate forming part of the spinous process fixation system of FIG. 9;

FIG. 10B is a plan view of the medial surface of the first plate of FIG. 9;

FIG. 11A is a plan view of the lateral surface of a second plate forming part of the spinous process fixation system of FIG. 9;

FIG. 11B is a plan view of the medial surface of the second plate of FIG. 9; and

FIG. 12 is a perspective view of one example of the coupling element forming part of the spinous process fixation system of FIG. 9.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The spinous process plate systems for spinal fusion disclosed herein boasts a variety of inventive features and components that warrant patent protection, both individually and in combination

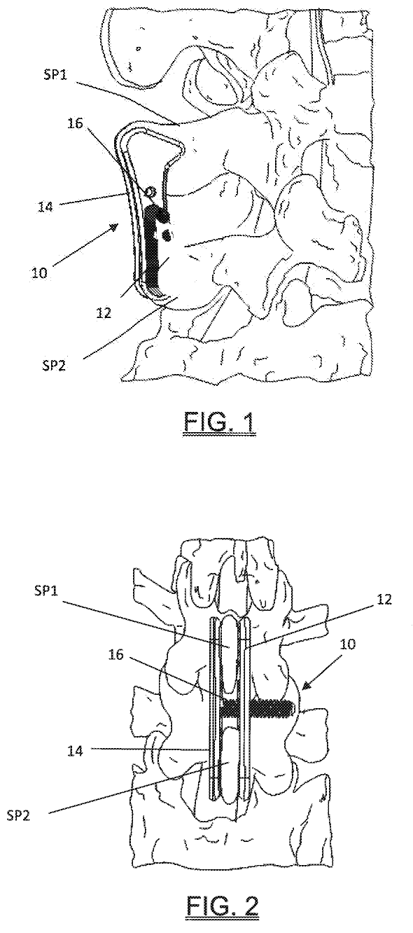

FIGS. 1-2 illustrate a spinous process fixation system 10 according to one embodiment of the present invention. The system 10 includes a first plate 12, a second plate 14, and a coupling element 16. The system 10 is designed to be attached to adjacent spinous processes SP1, SP2 of the spine for immobilizing the adjacent spinous processes SP1, SP2 to promote fusion therebetween. The system 10 may be used alone (that is, without any supplemental fusion devices, such as interbody fusion implants) as shown in FIGS. 1-2. Alternatively, the system 10 may be used with supplemental fixation devices (not shown). In either event, the system 10 allows fusion to occur between the adjacent spinous processes SP1, SP2 by maintaining them in an immobilized, locked relationship such that a boney bridge can form therebetween. The formation of the fusion bridge between the adjacent spinous processes SP1, SP2 may be augmented or facilitated by placing fusion-enhancing compounds between the spinous processes (such as, e.g. between the plates 12, 14), including but not limited to autologous bone, allograft bone, bone morphogenic protein (BMP), and/or any number of suitable biomaterials.

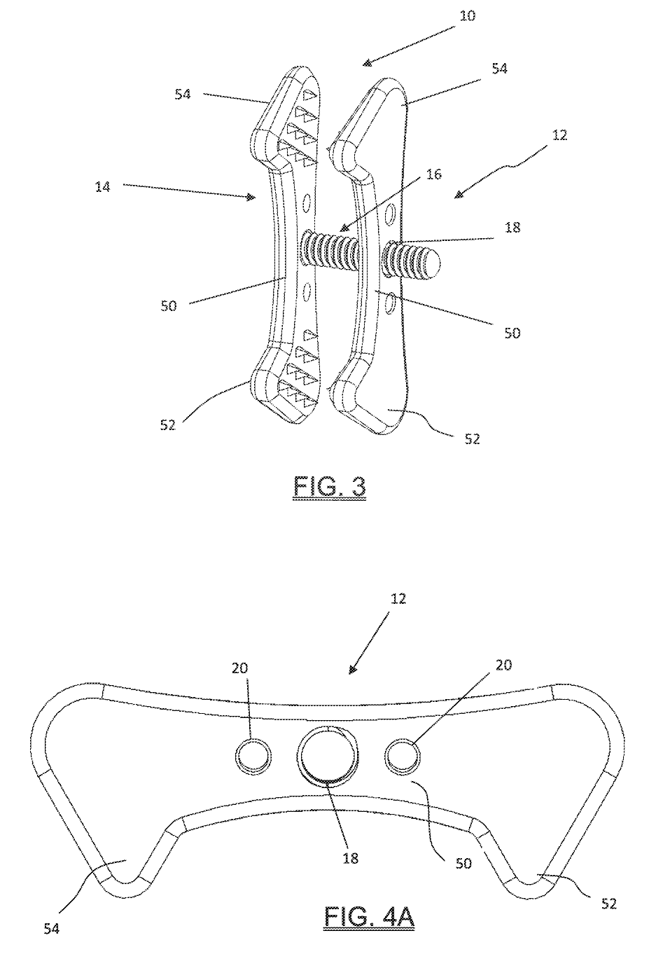

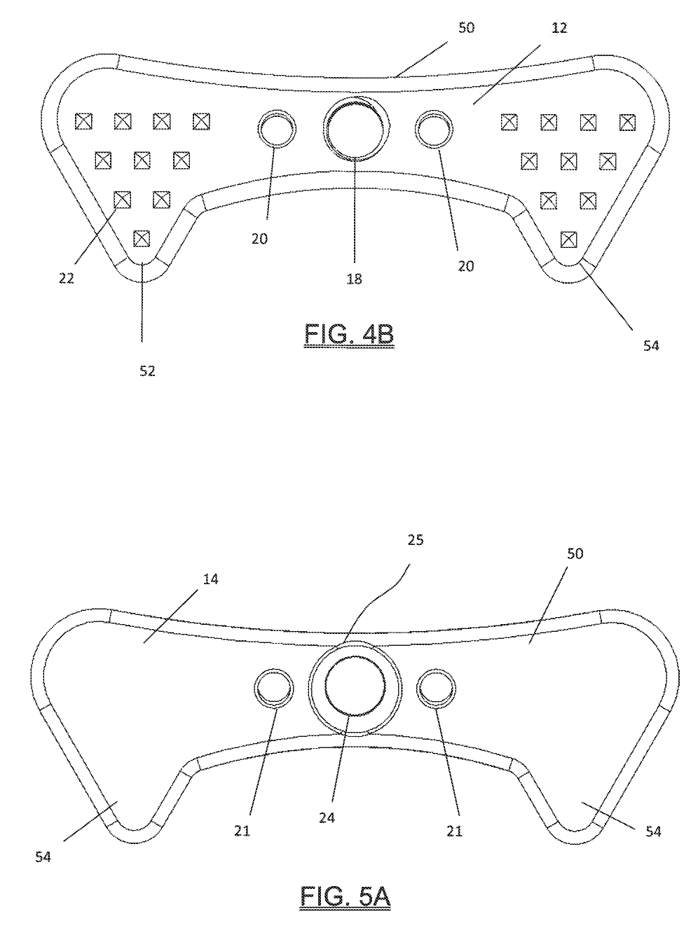

The specifics of the system 10 will now be described with reference to FIGS. 3-6. As shown in FIGS. 3-4B, the first plate 12 includes a central body portion 50 extending between a pair of end portions 52, 54. The central body portion 50 may have a generally curved perimeter and (as best viewed in FIGS. 4A-4B) has a width less than the width of the end portions 52, 54. The increased width of the end portions 52, 54 is designed to present a relatively large footprint on the adjacent spinous processes SP1, SP2, which helps in establishing a robust engagement therewith while avoiding protrusion beyond the spinous processes SP1, SP2. Although generally “hook” shaped in the embodiment shown, one of ordinary skill in the art will appreciate that the end portions 52, 54 may be provided in any number of suitable shapes including but not limited to generally rectangular, generally triangular, and generally rounded. As shown in FIG. 4B, this engagement may be augmented through the use of a plurality of spike elements 22 disposed on the medial facing surface of the end portions 52, 54. These spikes 22 are designed to become embedded in the lateral surface of the spinous processes SP1, SP2 when the system 10 is compressed in place as shown in FIG. 1.

The first plate 12 includes a central aperture 18 dimensioned to receive a distal portion of the threaded screw 16 as shown in FIGS. 1 and 3. More specifically, the central aperture 18 preferably includes an internal threading feature capable of threadedly cooperating with the threads of the screw 16. As will be described in greater detail below, the threaded engagement between the screw 16 and the first plate 12 allows the first plate 12 to be coupled to the second plate 14. The first plate 12 may also, according to one embodiment, include a pair of attachment apertures 20 positioned on either side of the central aperture 18. Each attachment aperture 20 is dimensioned to receive an extension element of an insertion tool (not shown). By way of example only, attachment aperture 20 may include, but not be limited to circular holes, indentations, or bosses that allow for the engagement of an insertion tool. The insertion tool may be used to hold and manipulate the plate 12 as needed to properly position it on the desired spinous processes SP1, SP2.

The first plate 12 may be constructed from any of a variety to suitable materials without departing from the scope of the invention, including but not limited to titanium, polymeric materials (e.g. plastics) carbon fiber, and/or any other biologically acceptable material. The first plate 12 may also be provided having any number of suitable dimensions without departing from the scope of the invention. For example, according to one embodiment, the width of the central body portion may range from 5 mm to 20 mm, the width of the end portions 52, 54 may range from 7.5 mm to 25 mm, the length of the central body portion 50 may range from 1 mm to 65 mm, the length of the end portions 52, 54 may range from 7.5 mm to 25 mm, and the thickness of the plate 12 may range from 1.5 mm to 15 mm. It will be appreciated, however, that these dimensions are provided as examples of those that may be employed with the system 10 of the present invention and any number of suitable modifications may be made depending upon a variety of factors without departing from the scope of the invention.

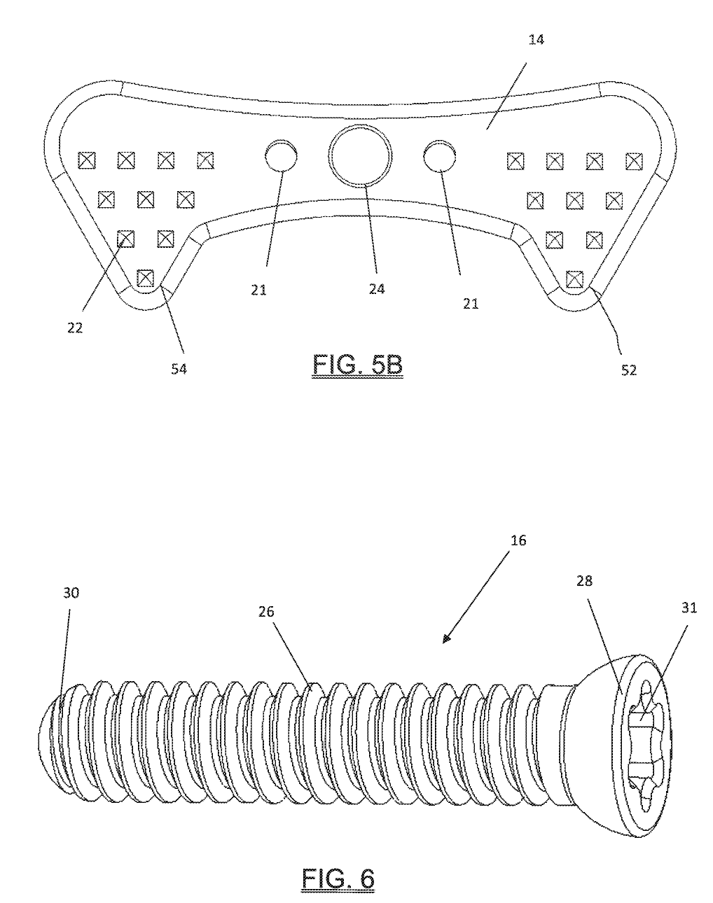

As shown in FIGS. 3 and 5A-5B, the second plate 14 includes the same general features as the first plate 12. The central body portion 50 extends between the end portions 52, 54. The central body portion 50 may have a generally curved perimeter and (as best viewed in FIGS. 5A-5B) has a width less than the width of the end portions 52, 54. The increased width of the end portions 52, 54 is designed to present a relatively large footprint on the adjacent spinous processes SP1, SP2, which helps in establishing a robust engagement therewith while avoiding protrusion beyond the spinous processes SP1, SP2. Although generally “hook” shaped in the embodiment shown, one of ordinary skill in the art will appreciate that the end portions 52, 54 may be provided in any number of suitable shapes including but not limited to generally rectangular, generally triangular, and generally rounded. As shown in FIG. 5B, this engagement may be augmented through the use of a plurality of spike elements 22 disposed on the medial facing surface of the end portions 52, 54. These spikes 22 are designed to become embedded in the lateral surface of the spinous processes SP1, SP2 when the system 10 is compressed in place as shown in FIG. 1.

The second plate 14 includes a central aperture 24 dimensioned to receive a proximal end of the threaded screw 16 as shown in FIGS. 1 and 3. More specifically, as best shown in FIG. 5A, the central aperture 24 includes a partially spherical surface 25 dimensioned to receive the partially spherical head 28 of the threaded screw 16 as will be described in detail in FIG. 6. The second plate 14 also, according to one embodiment, includes attachment apertures 20 positioned on either side of the central aperture 24. Each attachment aperture 20 is dimensioned to receive an extension element of an insertion tool (not shown). By way of example only, attachment aperture 20 may include, but not be limited to circular holes, indentations, or bosses that allow for the engagement of an insertion tool. The insertion tool may be used to hold and manipulate the plate 14 as needed to properly position it on the desired spinous processes SP1, SP2.

The second plate 14 may be constructed from any of a variety to suitable materials without departing from the scope of the invention, including but not limited to titanium, polymeric materials (e.g. plastics) carbon fiber, and/or any other biologically acceptable material. The second plate 14 may also be provided having any number of suitable dimensions without departing from the scope of the invention. For example, according to one embodiment, the width of the central body portion may range from 5 mm to 20 mm, the width of the end portions 52, 54 may range from 7.5 mm to 25 mm, the length of the central body portion 50 may range from 1 mm to 65 mm, the length of the end portions 52, 54 may range from 7.5 mm to 25 mm, and the thickness of the plate 12 may range from 1.5 mm to 15 mm. It will be appreciated, however, that these dimensions are provided as examples of those that may be employed with the system 10 of the present invention and any number of suitable modifications may be made depending upon a variety of factors without departing from the scope of the invention.

Referring now to FIG. 6, the coupling element 16 according to the first exemplary embodiment of the present invention is a threaded screw having a partially spherical head 28 and a shaft 30 extending therefrom with a threaded portion 26. The shaft 30 of the screw 16 is dimensioned to be passed through the central aperture 24 of the second plate 14 and then onward through the central aperture 18 of the first plate 12 to the point where the threaded portion 16 threadedly engages the threads along the interior of the central aperture 18 of the first plate 12. As this occurs, the partially spherical head 28 of the screw 16 will be drawn into the partially spherical surface 25 of the central aperture 24 of the second plate 14. The partially spherical head 28 has a larger diameter than the inner periphery of the central aperture 24 such that the head 28 cannot pass through the aperture 24 but rather cooperates with the surface 25. The screw 16 will thus draw the first plate 12 closer to the second plate 14 as the screw 16 is advanced through apertures 24, 18. This rotation may be accomplished through the use of any number of suitable driving devices, including but not limited to a screwdriver or Allen wrench capable of cooperating with corresponding features within the head 28 (e.g. Hexalobe-head grooves 31 in the screw head 28 shown in FIG. 6).

The screw 16 may be constructed from any of a variety to suitable materials without departing from the scope of the invention, including but not limited to titanium, polymeric materials (e.g. plastics) carbon fiber, and/or any other biologically acceptable material. The screw 16 may also be provided having any number of suitable dimensions without departing from the scope of the invention. For example, according to one embodiment, the width of the screw 16 may range from 3 mm to 10 mm, the length of the screw 16 may range from 15 nm to 50 mm, and the threaded portion 26 may have any number of suitable thread pitches. It will be appreciated, however, that these dimensions are provided as examples of those that may be employed with the system 10 of the present invention and any number of suitable modifications may be made depending upon a variety of factors without departing from the scope of the invention.

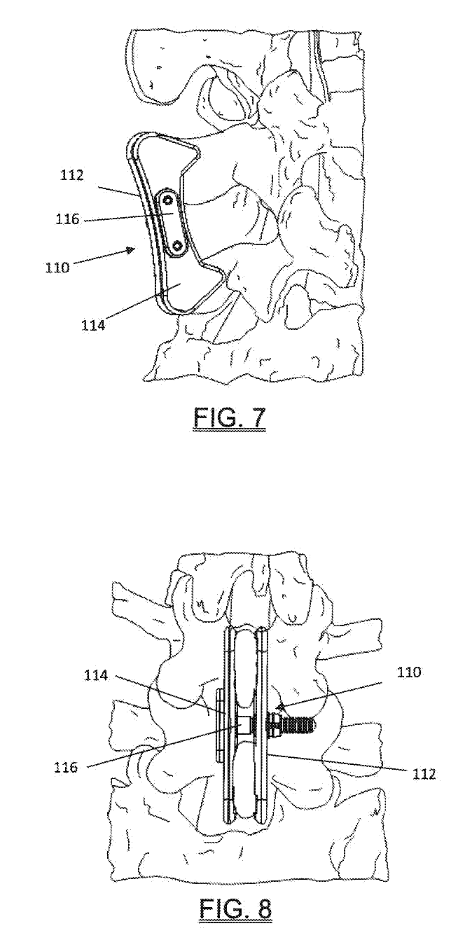

FIGS. 7-8 illustrate a spinous process fixation system 110 according to another embodiment of the present invention. The system 110 includes a first plate 112, a second plate 114, and a coupling element 116. The system 110 is designed to be attached to adjacent spinous processes SP1, SP2 of the spine for immobilizing the adjacent spinous processes SP1, SP2 to promote fusion therebetween. The system 110 may be used alone (that is, without any supplemental fusion devices, such as interbody fusion implants) as shown in FIGS. 7-8. Alternatively, the system 110 may be used with supplemental fixation devices (not shown). In either event, the system 110 allows fusion to occur between the adjacent spinous processes SP1, SP2 by maintaining them in an immobilized, locked relationship such that a boney bridge can form therebetween. The formation of the fusion bridge between the adjacent spinous processes SP1, SP2 may be augmented or facilitated by placing fusion-enhancing compounds between the spinous processes (such as, e.g. between the plates 112, 114), including but not limited to autologous bone, allograft bone, bone morphogenic protein (BMP), and/or any number of suitable biomaterials.

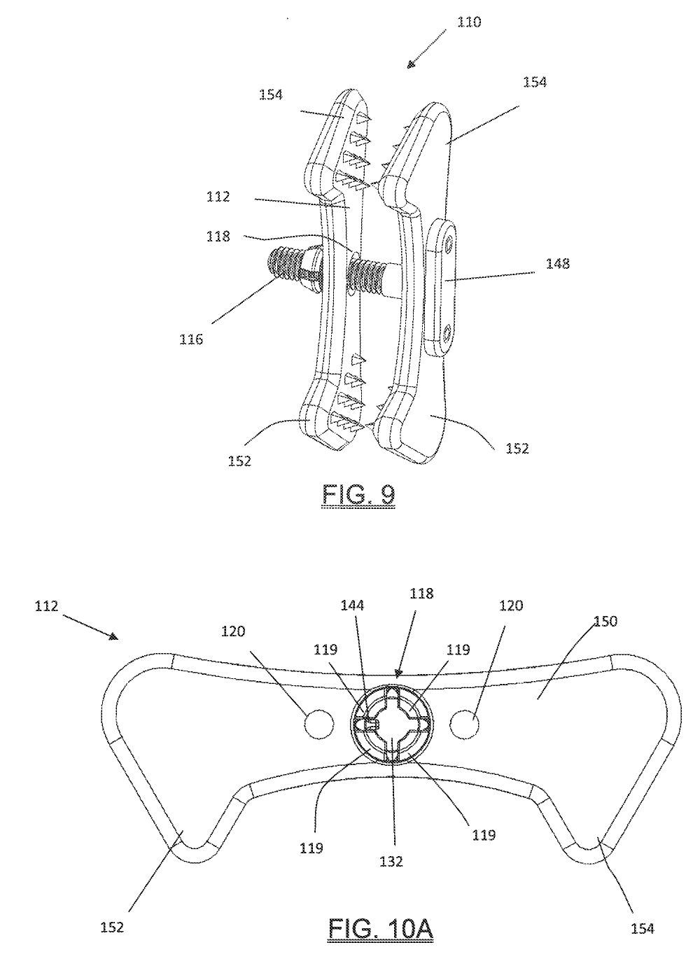

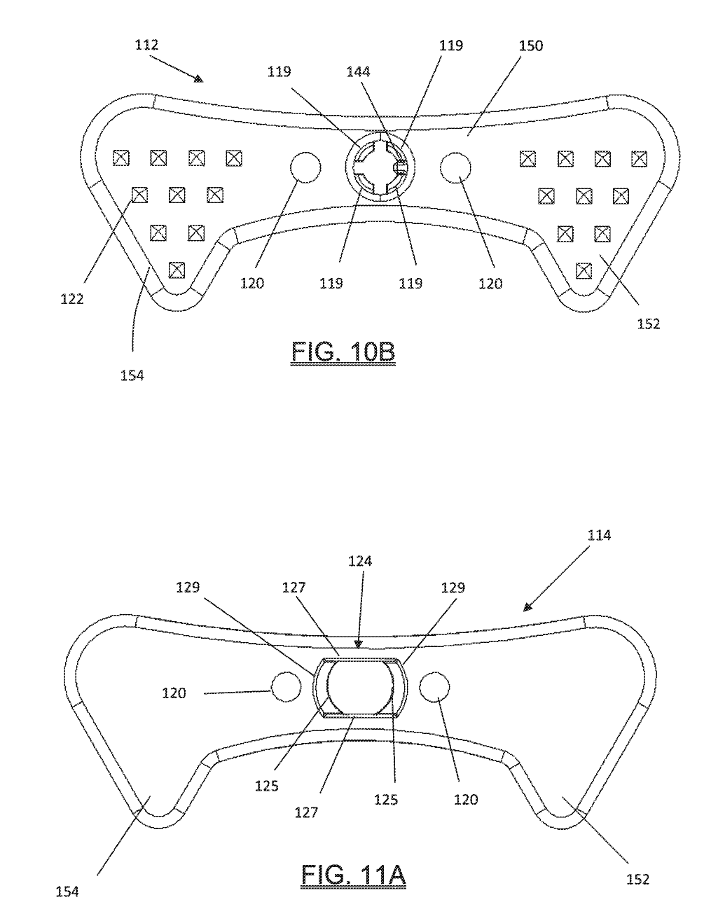

The specifics of the system 110 will now be described with reference to FIGS. 9-12. As shown in FIGS. 9 and 10A-10B, the first plate 112 includes a central body portion 150 extending between a pair of end portions 152, 154. The central body portion 150 may have a generally curved perimeter and (as best viewed in FIGS. 10A-10B) has a width less than the width of the end portions 152, 154. The increased width of the end portions 152, 154 is designed to present a relatively large footprint on the adjacent spinous processes SP1, SP2, which helps in establishing a robust engagement therewith while avoiding protrusion beyond the spinous processes SP1, SP2. Although generally “hook” shaped in the embodiment shown, one of ordinary skill in the art will appreciate that the end portions 152, 154 may be provided in any number of suitable shapes including but not limited to generally rectangular, generally triangular, and generally rounded. As shown in FIG. 10B, this engagement may be augmented through the use of a plurality of spike elements 122 disposed on the medial facing surface of the end portions 152, 154. These spikes 122 are designed to become embedded in the lateral surface of the spinous processes SP1, SP2 when the system 10 is compressed in place as shown in FIG. 7.

The first plate 112 includes a central aperture 118 dimensioned to receive a distal end of the ridged bolt 116 as shown in FIGS. 7 and 9. More specifically, as best viewed in FIG. 10A, the central aperture 118 preferably includes a series ridges or flanges 119 capable of cooperating with ridges along the bolt 116. As will be described in greater detail below, the ridged engagement between the bolt 116 and the first plate 112 allows the first plate 112 to be coupled to the second plate 114. The first plate 112, according to one embodiment, includes a rectangular boss anti-rotation feature 144 as shown in FIGS. 10A and 10B. A corresponding feature shown as a rectangular channel 146 in coupling element 116, engages the anti-rotation feature 144. This embodiment limits the rotation of the first plate 112 and second plate 114 relative to each other about the axis of the ridged bolt 116. Further, the first plate 112 also, according to one embodiment, includes a pair of attachment apertures 120 positioned on either side of the central aperture 118. Each attachment aperture 120 is dimensioned to receive an extension element of an insertion tool (not shown). The insertion tool may be used to hold and manipulate the plate 112 as needed to properly position it on the desired spinous processes SP1, SP2.

The first plate 112 may be constructed from any of a variety to suitable materials without departing from the scope of the invention, including but not limited to titanium, polymeric materials (e.g. plastics) carbon fiber, and/or any other biologically acceptable material. The first plate 112 may also be provided having any number of suitable dimensions without departing from the scope of the invention. For example, the width of the central body portion may range from 5 mm to 20 mm, the width of the end portions 152, 154 may range from 7.5 min to 25 mm, the length of the central body portion 150 may range from 1 mm to 65 mm, the length of the end portions 152, 154 may range from 7.5 mm to 25 mm, and the thickness of the plate 112 may range from 1.5 mm to 15 mm. It will be appreciated, however, that these dimensions are provided as examples of those that may be employed with the system 110 of the present invention and any number of suitable modifications may be made depending upon a variety of factors without departing from the scope of the invention.

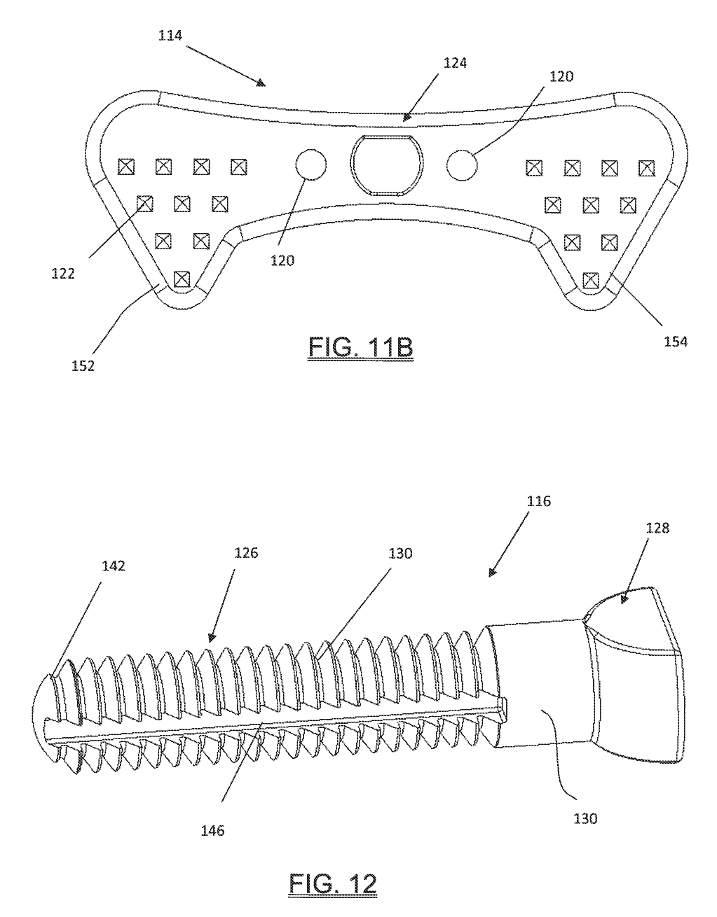

As shown in FIGS. 9 and 11A-11B, the second plate 114 includes the same general features as the first plate 112. The central body portion 150 extends between the end portions 152, 154. The central body portion 150 has a generally curved perimeter and (as best viewed in FIGS. 11A-11B) has a width less than the width of the end portions 152, 154. The increased width of the end portions 152, 154 is designed to present a relatively large footprint on the adjacent spinous processes SP1, SP2, which helps in establishing a robust engagement therewith while avoiding protrusion beyond the spinous processes SP1, SP. Although generally “hook” shaped in the embodiment shown, one of ordinary skill in the art will appreciate that the end portions 152, 154 may be provided in any number of suitable shapes including but not limited to generally rectangular, generally triangular, and generally rounded. As shown in FIG. 11B, this engagement may be augmented through the use of a plurality of spike elements 122 disposed on the medial facing surface of the end portions 152, 154. These spikes 122 are designed to become embedded in the lateral surface of the spinous processes SP1, SP2 when the system 110 is compressed in place as shown in FIG. 7.

The second plate 114 includes a central aperture 124 dimensioned to receive a proximal end of the ridged bolt 116 as shown in FIGS. 7 and 9. More specifically, as best shown in FIG. 11A, the central aperture 124 is a “truncated spherical” recess having straight sides 127 and semi-spherical end regions 129. The straight sides 127 and semi-spherical end regions 129 are dimensioned to receive the generally straight sides and semi-spherical end regions of the head 128 of the ridged bolt 116 as will be described in detail below. The second plate 114, according to one embodiment, includes retaining feature 148 that captures coupling member 116 inserted through central aperture 118. The second plate 114 also, according to one embodiment, includes attachment apertures 120 positioned on either side of the central aperture 124. Each attachment aperture 120 is dimensioned to receive an extension element of an insertion tool (not shown). The insertion tool may be used to hold and manipulate the plate 114 as needed to properly position it on the desired spinous processes SP1, SP2.

The second plate 114 may be constructed from any of a variety to suitable materials without departing from the scope of the invention, including but not limited to titanium, polymeric materials (e.g. plastics) carbon fiber, and/or any other biologically acceptable material. The second plate 114 may also be provided having any number of suitable dimensions without departing from the scope of the invention. For example, the width of the central body portion 150 may range from 5 mm to 20 mm, the width of the end portions 152, 154 may range from 7.5 mm to 25 mm, the length of the central body portion 150 may range from 1 mm to 65 mm, the length of the end portions 152, 154 may range from 7.5 mm to 25 mm, and the thickness of the plate 114 may range from 1.5 mm to 15 mm. It will be appreciated, however, that these dimensions are provided as examples of those that may be employed with the system 110 of the present invention and any number of suitable modifications may be made depending upon a variety of factors without departing from the scope of the invention.

FIG. 12 illustrates an example of a coupling element 116 according to one embodiment of the present invention. As shown, the coupling element 116 is a ridged bolt having a “truncated spherical” shaped head 128 and a shaft 130 extending therefrom with a ridged portion 126. The shaft 130 of the bolt 116 is dimensioned to be passed through the central aperture 124 of the second plate 114 and then onward through the central aperture 118 of the first plate 112 to the point where the ridged portion 116 matingly engages the ridges or flanges 119 along the interior of the central aperture 118 of the first plate 112. As this occurs, the truncated spherical head 128 of the bolt 116 will be advanced into the central aperture 124 of the second plate 114. The truncated spherical head 128 has a larger diameter than the inner periphery of the central aperture 124 such that the head 128 cannot pass through the aperture 124 but rather cooperates in a “keyed” fashion with the central aperture 124. Specifically, straight sides 131 and semi-spherical portions 133 of the head 124 cooperate with the straight sides 127 and semi-spherical end regions 129, respectively, of the aperture 124. The bolt 116 will thus couple the first plate 112 to the second plate 114 as the bolt 116 is advanced axially into engagement with the ridges or flanges 119 of the first plate 112. In one embodiment, the ridges or flanges 119 are dimensioned such that the ridges 126 of the bolt 116 pass relatively easily through the aperture 118 towards the lateral facing surface of the first plate 112 but relatively difficulty in the opposite direction. In this manner, the first plate 112 and second plate 114 will be coupled in a secure manner on adjacent sides of the spinous processes SP 1, SP2. Any of a variety of tools may be used to remove the bolt 116 from engagement with the flanges 119 of the central aperture 118 so as to disengage the first plate 112 from the second plate 114.

The bolt 116 may be constructed from any of a variety to suitable materials without departing from the scope of the invention, including but not limited to titanium, polymeric materials (e.g. plastics) carbon fiber, and/or any other biologically acceptable material. The bolt 116 may also be provided having any number of suitable dimensions without departing from the scope of the invention. For example, in one exemplary embodiment, the width of the bolt 116 may range from 3 mm to 11 mm, the length of the bolt 116 may range from 15 mm to 50 mm, and the ridged portion 126 may range from 5 mm to 47 mm. It will be appreciated, however, that these dimensions are provided as examples of those that may be employed with the system 110 of the present invention and any number of suitable modifications may be made depending upon a variety of factors without departing from the scope of the invention.

The embodiments described herein are intended to rigidly fix two spinous processes relative to one another. The devices 10, 110 may be implanted via a traditional “open” procedure or a minimally invasive procedure. In a minimally invasive procedure, the devices 10, 110 may be implanted generally posteriorly through a single incision (e.g. where the first plates 12, 112 and second plates 14, 114 are passed through the same incision) or multiple incisions (e.g. where the first plates 12, 112 are passed through one incision and the second plates 14, 114 are passed through a second incision). During a uni-portal introduction, the surgeon may pass both the first plate 12, 112 and the second plate 14, 114 into position on either side of adjacent spinous processes SP1, SP2 at the same time. During a bi-portal introduction, the surgeon may first insert the first plate 12, 112 to engage one side of the spinous processes SP1, SP2 and then insert the second plate 14, 114 against the spinous processes SP1, SP2. In either event, the surgeon can adjust the position of the end portions 52, 54, 152, 154 on the first plate 12, 112 and second plate 14, 114 so that the spike members 22, 122 are engaged into the spinous processes SP1, SP2. At this point, compression instrumentation may be applied to press the plates toward each other, whereupon the spikes enter the spinal processes SP1, SP2. Following the full seating of the plates on the spinal processes SP1, SP2, the screw 16 and bolt 116 are tightened using any number of suitable instruments. When the surgeon is satisfied with the degree to which the first plate 12, 112 and second plate 14, 114 are locked together (e.g. if a desired torque level is applied to the screw 16), then the site may be closed up, completing the stabilization procedure.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the invention is to cover all modifications, equivalents, and alternatives falling within the spirit and scope of the invention as defined herein.

What is claimed is:

1. A plating system for fusing a first spinous process and a second spinous process, said first spinous process forming part of a first vertebra and said second spinous process forming part of a second vertebra, each of said spinous processes including a first side and a second side, said plating system comprising:
a first plate having a first surface for contacting said first sides of said first and second spinous processes, a second surface opposite said first surface, a first end portion, a second end portion, and a generally elongated body portion extending between said first and second end portions, wherein at least one of said first and second end portions has a width dimension greater than a width dimension of said body portion;
a second plate having a first surface for contacting second sides of said first and second spinous processes, a second surface opposite said first surface, a first end portion, a second end portion, and a generally elongated body portion extending between said first and second end portions, wherein at least one of said first and second end portions has a width dimension greater than a width dimension of said body portion; and
an elongated connector element extending through said first and second plates, said connector element adjustably attached to at least one of said first and second plates.

2. The plating system of claim 1, wherein said first surface of said first plate includes a first contact region dimensioned to engage at least a portion of said first side of said first spinous process and a second contact region dimensioned to engage at least a portion of said first side of said second spinous process, and said first surface of said second plate includes a first contact region dimensioned to engage at least a portion of said second side of said first spinous process and a second contact region dimensioned to engage at least a portion of said second side of said second spinous process.

3. The plating system of claim 2, wherein said first contact region of said first plate is located on said first end portion of said first plate, said second contact region of said first plate is located on said second end portion of said first plate, said first contact region of said second plate is located on said first end portion of said second plate, and said second contact region of said second plate is located on said second end portion of said second plate.

4. The plating system of claim 3, wherein said first and second contact regions of said first and second plates each include a plurality of anti-migration features to secure the first and second plates to said first and second spinous processes.

5. The plating system of claim 4, wherein said anti-migration features comprise spikes.

6. The plating system of claim 1, wherein at least one of said first and second end portions of said first and second plates is at least one of generally rectangular, generally triangular, and generally rounded.

7. The plating system of claim 1, wherein said connector element includes a shaped head element and a shaft element.

8. The plating system of claim 7, wherein said shaft element is threaded.

9. The plating system of claim 8, wherein said first plate includes a threaded aperture extending between said first and second surfaces, said threaded aperture dimensioned to threadedly receive a distal portion of said shaft element.

10. The plating system of claim 7, wherein said shaft element includes a plurality of non-threaded ridges.

11. The plating system of claim 10, wherein said first plate includes an aperture extending between said first and second surfaces, said aperture dimensioned to receive a distal portion of said shaft element.

12. The plating system of claim 11, wherein said aperture includes at least one flange extending therefrom, said flange dimensioned to engage said plurality of ridges to adjustably secure said connector element to said first plate.

13. The plating system of claim 11, wherein said connector element further includes an elongated recess extending substantially the length of said shaft element.

14. The plating system of claim 13, wherein said aperture further includes an anti-rotation element dimensioned to engage said elongated recess to prevent rotation of said connector element.

15. The plating system of claim 7, wherein said second plate includes an aperture extending between said first and second surfaces, said aperture dimensioned to receive said shaped head element.

16. The plating system of claim 15, wherein said aperture is threaded.

17. The plating system of claim 15, wherein said aperture has a shape corresponding to said shaped head element.

18. The plating system of claim 1, wherein at least one of said first and second end portions of said first and second plates has a width dimension within a range from 7.5 millimeters to 25 millimeters.

19. The plating system of claim 1, wherein said elongated body portions of said first and second plates have a width dimension within a range from 5 millimeters to 20 millimeters.

20. A method of immobilizing a first spinous process forming part of a first vertebra relative to a second spinous process forming part of a second vertebra, said method comprising:
attaching a first plate to first sides of said first and second spinous processes, said first plate including a first end portion, a second end portion, and a generally elongated body portion extending between said first and second end portions, wherein at least one of said first and second end portions has a width dimension greater than a width dimension of said body portion;
attaching a second plate to second sides of said first and second spinous processes, said second plate including a first end portion, a second end portion, and a generally elongated body portion extending between said first and second end portions, wherein at least one of said first and second end portions has a width dimension greater than a width dimension of said body portion; and
advancing an elongated connector element through said first and second plates until said first and second plates are secured to said first and second spinous processes, said connector element adjustably attached to at least one of said first and second plates.
